(19) United States
(12) Patent Application Publication
(10) Pub. No.: US 2014/0309738 A1
(43) Pub. Date: Oct. 16, 2014

(54) Membrane-Scaffold Composites for Tissue Engineering Applications

(71) Applicant: The Board of Trustees of the University of Illinois, Urbana, IL (US)

(72) Inventors: Brendan A. Harley, Urbana, IL (US); Steven R. Caliari, Philadelphia, PA (US); Manuel Alejandro Ramirez Garcia, Rochester, NY (US)

(21) Appl. No.: 14/122,337

(22) PCT Filed: Jun. 1, 2012
(86) PCT No.: PCT/US2012/040368

§ 371 Date: Jun. 23, 2014

Related U.S. Application Data
(60) Provisional application No. 61/491,999, filed on Jun. 1, 2011.

(51) Int. Cl. A61F 2/08

(52) U.S. Cl. CPC A61F 2/08
USPC 623/13.11; 264/28; 425/506

(57) ABSTRACT

Collagen-glycosaminoglycan membrane shell scaffold core composites for connective tissue engineering that avoids aspects of the typical tradeoff between mechanical properties (i.e. modulus, failure strength) and bioactivity (i.e., permeability and porosity) for porous tissue engineering scaffolds. The relative density of the collagen glycosaminoglycan scaffold core can be about 0.5 to about 0.95 while the membrane shell can be about 0.001 to 25 about 0.2. The core-shell composite can be tubular and the composite can have a diameter of about 1 mm to about 20 mm. The collagen glycosaminoglycan membrane shell can be perforated with about 25 to about 1000 micrometers openings or alternatively can be embossed with any range of pattern features from about 25 to about 1000 micrometers in size. The porous collagen glycosaminoglycan scaffold core can be populated with cells such as adult or embryonic stem cells, tenocytes, osteoblasts, nerve cells, cardiac cells, myocytes, fibroblasts or combinations thereof.

### PRIORITY

This application claims the benefit of U.S. Provisional patent application Ser. No. 61/491,999, filed on Jun. 1, 2011, which is incorporated herein by reference in its entirety.

### GOVERNMENT INTEREST

This work was supported by the Chemistry-Biology Interface Training Program NIH NIGMS T32GM070421 (SRC) and the U.S. Department of Energy under grants DE-FG02-07ER46453 and DE-FG02-07ER46471. The United States government has certain rights in this invention.

### BACKGROUND OF THE INVENTION

Tendons are specialized connective tissues that transmit tensile loads between bone and muscle. Their functional capacity derives from a unique extracellular matrix (ECM) composed primarily of type I collagen arranged in a highly organized hierarchy of parallel, cross-linked fibrils [1,2]. Tendon and ligament injuries are common among both recreational and elite athletes as well as the elderly. Of the near 35 million musculoskeletal injuries in the US every year, approximately 50% involve tendons and ligaments with a cost to the US health care industry in the tens of billions of dollars per year [3]. The most serious injuries require surgical intervention; such tendon and ligament injuries are responsible for hundreds of thousands of surgical procedures each year in the US [2,3].) One of the key challenges of orthopedic tissue engineering is to create biomaterials that can support tissue regeneration while remaining mechanically competent. Due to the need for mechanical competence, the most common biomaterial designs for tendon and ligament tissue engineering are electrospun polymer mats [4-6] and woven fibrous materials [7,8]. While these constructs can promote cell alignment and be designed with tensile moduli approaching the level of tendon, they are dense substrates that permit limited cell penetration compared to the traditional tissue engineering target for a fully three-dimensional biomaterial structure. As an alternative, porous scaffold biomaterials typically have highly tunable 3D microstructural features, show significantly heightened levels of permeability, and can be fabricated from a range of natural, biodegradable materials. However, the relative density (ρ*/ρₛ; 1% porosity where ρ* is the density of the porous foam and ρₛ is the density of the solid it is constructed from) of these porous scaffolds differentially affects a number of critical scaffold properties. Notably, increasing scaffold ρ*/ρₛ increases both its specific surface area, impacting cell attachment, and its elastic modulus, which varies with (ρ*/ρₛ)^{2 [}9-12]. However, increasing scaffold ρ*/ρₛ also increases steric hindrance to cell penetration and, critically, reduces scaffold permeability [13], negatively impacting cell penetration into the porous structure and long-term survival. Due to the high porosity (>90%) typically required for most tissue engineering scaffolds to adequately support cell bioactivity [14], these materials are often orders of magnitude too soft for orthopedic applications such as for tendon. Mechanical stimulation of cell-seeded scaffold constructs has been used to marginally improve construct mechanical properties, however not to the level of native tendon or ligament [15-18].

Current tissue engineering approaches for tendon defects require improved biomaterials to balance microstructural and mechanical design criteria. Collagen-glycosaminoglycan (CG) scaffolds have shown considerable success as in vivo regenerative templates and in vitro constructs to study cell behavior. While these scaffolds possess many advantageous qualities, their mechanical properties are typically orders of magnitude lower than orthopedic tissues such as tendon.

### SUMMARY OF THE INVENTION

In one embodiment, the invention provides a core-shell composite comprising a porous collagen glycosaminoglycan scaffold core and a collagen glycosaminoglycan membrane shell having a higher density than the core, wherein the membrane shell is cross-linked to the core. The relative density of the collagen glycosaminoglycan scaffold core can be about 0.5 to about 0.95. The relative density of the collagen glycosaminoglycan membrane shell can be about 0.001 to about 0.2. The core-shell composite can be tubular and the composite can have a diameter of about 1 mm to about 20 mm. The collagen glycosaminoglycan membrane shell can be periodically perforated with about 25 to about 1000 μm openings or alternatively can be embossed with any range of pattern features from about 25 to about 1000 μm in size. The porous collagen glycosaminoglycan scaffold core can be populated with cells. The scaffold core and/or the membrane shell can be isotropic or anisotropic. The cells present in the scaffold core can be adult or embryonic stem cells, tenocytes, osteoblasts, nerve cells, cardiac cells, myocytes, fibroblasts or combinations thereof.

Another embodiment of the invention provides a method of making a core-shell composite. The method comprises making a collagen glycosaminoglycan membrane shell by placing a collagen glycosaminoglycan suspension on a solid surface and allowing the suspension to dry or partially dry to form a collagen glycosaminoglycan membrane shell. The collagen glycosaminoglycan membrane shell is placed in a mold so that the longitudinal surfaces of the mold are covered with the membrane shell, leaving a center core portion of the mold open. A collagen glycosaminoglycan suspension is placed in the center core portion of the mold and the mold in a pre-cooled freeze dryer. Ice crystals are sublimated to form an non-cross-linked composition. The non-cross-linked composition is removed from the mold and the composition is cross-linked to form a core-shell composite.

Still another embodiment of the invention provides a method of inducing growth of tissue having an aligned structure. The method comprises contacting a core-shell composite of the invention with one or more cell types that are capable of forming tissue having an aligned structure and allowing the cells to grow such that growth of tissue having an aligned structure is induced. The tissue having an aligned structure can be bone tissue, cardiac tissue, muscle tissue, peripheral nerve tissue, central nerve tissue, connective tissue, ligament tissue, meniscus tissue, rotator cuff tissue, skin tissue, cartilage tissue, or tendon tissue. The cells can be adult or embryonic stem cells, tenocytes, osteoblasts, nerve cells, cardiac cells, myocytes, fibroblasts or combinations thereof.

Yet another embodiment of the invention provides a method of treating a tissue or defect in a subject in need thereof. The method comprises administering one or more of the core-shell composites of the invention to the subject, thereby treating the tissue defect. The tissue defect can be a defect of bone tissue, cardiac tissue, muscle tissue, peripheral nerve tissue, central nerve tissue, connective tissue, ligament tissue, meniscus tissue, rotator cuff tissue, skin tissue, cartilage tissue, or tendon tissue. The core-shell composite can be seeded with one or more types of cells prior to administering the core-shell composite to the subject.

Even another embodiment of the invention provides a kit comprising a core-shell composite of the invention wherein the core-shell composite is immersed in a medium or is dried or partially dried and present in a storage container suitable for preserving the core-shell composite. The core-shell composite can be seeded with one or more types of cells.

Taking inspiration from mechanically efficient core-shell composites in nature such as plant stems and porcupine quills, membrane shell-scaffold core CG composites that display high bioactivity and improved mechanical integrity have been created. These composites feature integration of a low density, anisotropic CG scaffold core with a high density, CG membrane shell. CG membrane shells are fabricated via an evaporative process that allows separate tuning of membrane thickness and elastic moduli and that are isotropic in-plane. The membrane shells are then integrated with an anisotropic CG scaffold core via freeze-drying and subsequent cross-linking. Increasing the relative thickness of the CG membrane shell increases the composite tensile elastic modulus by as much as 2 or 3 orders of magnitude. The invention proves an effective, biomimetic approach for balancing strength and bioactivity requirements of porous scaffolds for tissue engineering applications.

### BRIEF DESCRIPTION OF THE DRAWINGS

FIG. 1 shows a diagram of a core-shell CG biomaterial composite that integrates a high density (high tensile strength) isotropic CG membrane with a low density (highly porous) anisotropic CG scaffold. d is the diameter of the scaffold core. t is the thickness of the membrane shell.

FIG. 2A shows cross-sectional SEM image of a CG membrane illustrating the dense, layered fibrillar organization. Scale bar: 10 μm. FIG. 2B shows that CG membranes can be produced over a wide range of thicknesses (23-240 μm) with consistent relative density (0.75-0.80) (n=17). Error bars: Mean±SD.

FIG. 3A shows the effect of increasing the intensity of cross-linking on the tensile modulus of 1% w/v 1×CG membranes (n=6). FIG. 3B shows the comparison of stress-strain curves for each 1% w/v 1×CG membrane variant. Increasing cross-linking treatments: NC (non-cross-linked), DHT (dehydrothermal cross-linked), EDAC 1:1:5 and EDAC 5:2:1 (EDAC:NHS:—COOH ratios). FIG. 3C shows both aligned CG scaffolds have significantly higher tensile moduli than isotropic CG controls (adapted from [1]; n=6). No significant difference was observed in tensile modulus between aligned CG scaffolds fabricated at two different pore sizes (243 mm, 55 mm). Error bars: Mean±SD.

FIG. 4A shows a SEM image of a representative longitudinal CG scaffold section, which shows the aligned, elongated pore structure present in the anisotropic cores of the core-shell composites. The vertical white arrow indicates the direction of heat transfer during directional solidification. Scale bar: 100 μm. FIG. 4B shows an SEM image of a representative transverse section through the scaffold displays the round, isotropic pore structure. Scale bar: 100 μm. FIG. 4 shows an SEM image of transverse plane of the scaffold-membrane composite showing the integration of the two regions. Scale bar: 1 mm.

FIG. 5A shows the tensile modulus of the core-shell CG composites increases significantly with membrane thickness. The experimentally measure tensile modulus (n=6, black squares) compares favorably to the predicted composite modulus obtained from layered composites theory (black line). The close agreement is indicative of integration of the membrane shell with scaffold core. Error bars: Mean±SD. FIG. 5B shows representative stress-strain curves for the series of core-shell composites with increasing shell thickness.

FIG. 6A shows core-shell CG composites (Membrane) support significantly higher tenocytes (TC) numbers at day 1 (n=6) and similar cell number at days 7 and 14 (n=6) compared to CG scaffolds alone (No membrane). Both groups show large increases in TC number from day 1 to day 7 and day 7 to day 14. FIG. 6B shows CG scaffolds alone (No membrane) display higher TC metabolic activity at day 1 (n=18), significantly higher metabolic activity at day 7 (n=12), and higher TC metabolic activity at day 14 (n=6) compared to the core-shell CG composites (Membrane). However, both groups show large increases in TC metabolic activity from day 1 to day 7 and subsequent maintenance of metabolic activity through day 14. Error bars: Mean±SD.

### DETAILED DESCRIPTION OF THE INVENTION

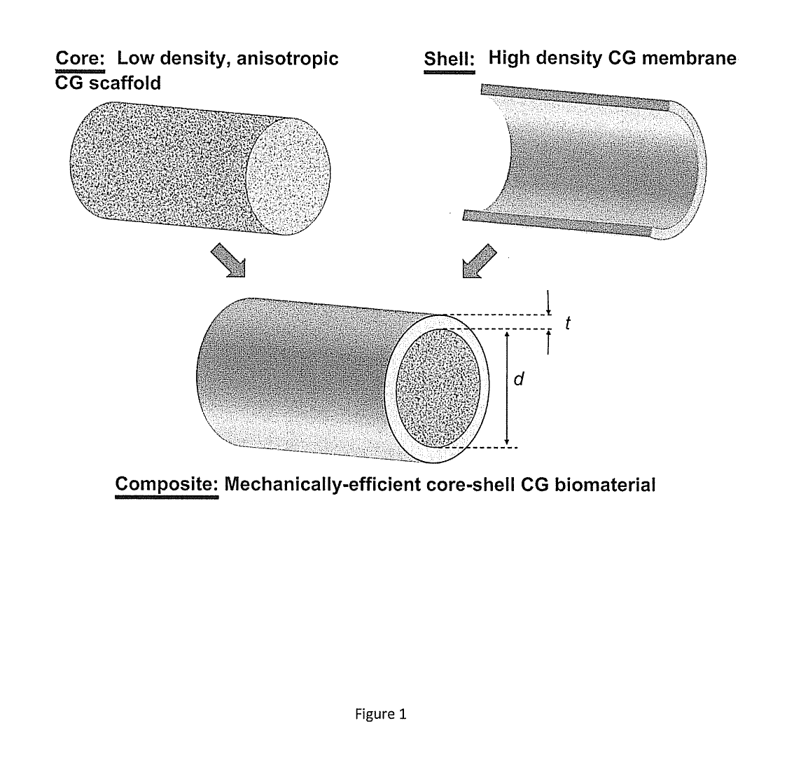

The invention provides a new class of core-shell CG biomaterial composites that integrate a high density (high tensile strength) isotropic CG membrane with a low density (highly porous) anisotropic CG scaffold (FIG. 1). CG membranes are integrated with aligned CG scaffolds in a manner to maintain adequate permeability to support cell proliferation and bioactivity. Such a composite biomaterial improves regenerative capacity by significantly improving construct mechanical integrity while still presenting a highly porous scaffold microstructure containing aligned contact guidance cues providing significant value for musculoskeletal tissue engineering applications.

### Collagen-Glycosaminoglycan Membrane Shells

Membrane shells of the invention are comprised of any type of collagen (e.g., collagen type I, II, III, IV, V or VI-XXVIII or combinations thereof) and one or more glycosaminoglycans (e.g., chondroitin 6-sulfate, heparin sulfate, heparin, dermatan sulfate, keratin sulfate, hyaluronic acid, or combinations thereof). The membrane shells can be fabricated from between about 0.5 wt % to about 5 wt % collagen-glycosaminoglycan suspension and collagen:glycosaminoglycan ratios of about 1:1 to about 20:1 can be used. A membrane shell can be isotropic. That is, the membrane shell contains uniform or randomly sized open cell structure. Alternatively, the membrane shell can be anisotropic, that is, the membrane shell is directionally dependent. For example, the membrane shells can have aligned tracks or ellipsoidal pores.

A membrane shell can be about 10 to about 500 μM thick or any range or value between about 10 to about 500 μM thick, for example about 20 to about 250 μM thick.

The relative density of a membrane shell can be about 0.5 to about 0.95, or any range or value between about 0.5 to about 0.95, for example about 0.75 to about 0.8. The porosity of a membrane shell can be about 5% to about 50% (or any range or value between about 5% to about 50%, for example about 20% to about 25%.

The tensile modulus can be isotropic or anisotropic in plane depending on fabrication methods. In one embodiment of the invention, the dry tensile modulus of a membrane shell in the perpendicular direction (e.g., perpendicular to the length of a cylindrical, completed membrane shell scaffold core composite) can be about 250 to about 1000 MPa or any range or value between about 250 to about 1000 MPa, for example about 585 to about 685 MPa.

The dry tensile modulus of a membrane in the parallel direction (e.g., parallel to the length of a cylindrical, completed membrane shell scaffold core composite) can be about 250 to about 1000 MPa or any range or value between about 250 to about 1000 MPa, for example about 670 to about 715 MPa.

The tensile modulus of a hydrated, cross-linked membrane shell can be about 10 to about 500 MPa or any range or value between about 10 to about 500 MPa, for example about 25 to about 35 MPa. The tensile modulus depends upon cross-linking and can vary from the presented values.

Optionally, a CG membrane shell can be periodically perforated with large (about 25 to about 1000 μm, or any range or value between about 50 to about 1000 μm, for example from about 250 to about 750 μm, for example about 500 μm) openings to facilitate radial cell penetration.

### Collagen Glycosaminoglycan Scaffold Cores

Scaffold cores of the invention are comprised of any type of collagen (e.g., collagen type I, II, III, IV, V or VI-XXVIII or combinations thereof) and one or more glycosaminoglycans (e.g., chondroitin 6-sulfate, heparin sulfate, heparin, dermatan sulfate, keratin sulfate, hyaluronic acid or combinations thereof). The scaffold cores can be fabricated from between about 0.5 wt % to about 5 wt % collagen-glycosaminoglycan suspension and collagen:glycosaminoglycan ratios of about 1:1 to about 20:1 can be used.

A scaffold core can be any of a variety of shapes including sheets, slabs, cylinders, tubes with any cross-sectional shape (e.g., circular, square, hexagonal), spheres, or beads. A scaffold core can also be provided in a shape that provides natural contours of a body part, e.g., a ligament, a tendon, a bone, a meniscus, etc. Where the scaffold core is in cylindrical, tube or sphere shape, the diameter of the scaffold core can be about 1 to about 25 mm, or any range or value between about 1 and about 25 mm, for example about 6-8 mm. A scaffold core can have a length of about 5 to about 500 mm, or any range or value between about 5 to about 500 mm, such as about 10 to about 50 mm.

The relative density (ρ*/ρₛ) of a scaffold core is less than that of the membrane shell and can be about 0.001 to about 0.2 or any range or value between about 0.001 to about 0.2, for example about 0.004 to about 0.02, or about 0.01 to about 0.02, for example, 0.015, 0.016 or 0.017. Scaffold relative density is an important parameter because it can influence on construction mechanics, permeability, specific surface area, and potential for steric hindrance. Additionally, the relative density can influence cell proliferation with the scaffold, metabolic activity of cells, contractive capacity, soluble collagen synthesis by cells, e.g., tenocytes or fibroblasts. Cells such as fibroblasts and tenocytes can buckle scaffold struts and deform local strut microarchitecture. Scaffold relative density can impact cell caused contraction and strut buckling. Higher density scaffolds provide the greatest resistance to cell mediated contraction.

Scaffold permeability is an important parameter that dictates the diffusion and exchange of soluble factors, nutrients and waste throughout the scaffold. Permeability can be about 1×10⁻¹² m² to about 1×10⁻⁰⁵ m² (or any range or value between about 1×10⁻¹² m² to about 1×10⁻⁵ m²), with a trend toward about 1×10⁻¹² m² as compressive strain increases.

A scaffold core can be geometrically anisotropic (i.e., the core is directionally dependent) with aligned pore structure or isotropic (containing a uniform or randomly-sized open cell structure). For example, anisotropic scaffolds can have aligned tracks or ellipsoidal pores that mimic elements of native connective tissue anisotropy such as tendons and ligaments. Anisotropic scaffolds have aligned ellipsoidal pore tracks in the longitudinal plane. Pores in the transverse plane maintain a rounded morphology. Therefore, an anisotropic scaffold, for example a cylindrical scaffold, has a significantly greater pore aspect ratio in the longitudinal than in the transverse planes meaning that the pores are elongated in the direction of the scaffold longitudinal axis. For example, the pore aspect ratio for the non-aligned transverse plane can be about 0.08 to about 2.0 or any range or value between about 0.08 and 2.0, for example from about 1.07 to about 1.22, or about 1.14, 1.15, or 1.16. The pore aspect ratio in the longitudinal direction can be about 3.0 to about 0.8 or any range or value between about 5.0 and 0.8, for example from about 2.01 to about 1.3, or about 1.8 to about 1.3. In one embodiment of the invention, the pore size and pore aspect ratios are similar throughout the entire scaffold.

Transverse pore size can be about 500 to about 20 μm, or any range or value between about 500 to about 20 μm, for example about 400 to about 200 μm, from about 313 to about 194 μm, or from about 267 to about 194 μm.

For isotropic scaffold cores the pore aspect ratio can be about 0.08 to about 2.0 or any range or value between about 0.08 and 2.0, for example from about 1.07 to about 1.22, or about 1.14, 1.15, or 1.16. The pore size can be about 500 to about 20 μm, or any range or value between about 500 to about 20 μm, for example about 400 to about 200 μm, from about 313 to about 194 μm, or from about 267 to about 194 μm.

In one embodiment of the invention a scaffold of the invention is populated by cells. The cells can be one or more types of cells such as fetal, embryonic, cord, mesenchymal, or hematopoietic stem cells; stem cells derived from muscle, skin, bone marrow, cardiac, synovium, or adipose tissue; fibroblasts; endothelial cells; osteoblasts; osteoclasts; osteocytes; tenocytes; non-stem cells differentiated from stem cell such as fetal, embryonic, cord blood, mesenchymal or hematopoietic stem cells; osteoblast progenitor cells; osteoblast-like cells; chondrocytes; and myocytes. The cells can be distributed equally throughout the scaffold or can be unequally distributed in the scaffold (e.g., different densities or types of cells in different portions of the scaffold). The cells can be derived from the subject to be treated (autologous source) or from allogeneic sources or xenogeneic sources such as embryonic stem cells or other cells. Optionally, the cells do not induce an immunogenic reaction in the subject. Cells can show longitudinal alignment in the scaffold, e.g., aligned between about −10° and +10° of the longitudinal (axial) axis of the scaffold core or can be distributed in a manner so as to not exhibit a preferred orientation, e.g., between −90° and +90° of the longitudinal axis of the scaffold core.

Cells, such as tenocytes and fibroblasts, are known to differentiate in 2-dimensional cell culture (e.g., cell culture flasks). Significant increases in cell proliferation as well as the expression of certain factors, which are indicative of differentiation, can occur in the 3-dimensional scaffolds of the invention as compared to 2-dimensional culture systems. For example, equine tenocytes can show significant increases of expression of transcription factor scleraxis (SCX), the glycoprotein tenascin-C (TNC), collagen (i.e. COL3A1), and matrix metalloproteinase 3 (MMP3) in a 3-dimensional scaffold of the invention as compared to 2-dimensional cell culture. Higher levels of COL3A1, SCX, TNC, and MMP3 indicate healthier tissue than lower levels of these markers. Additionally, expression levels of MMP1 and MMP13 of equine tenocytes were lower in a 3-dimensional scaffold of the invention as compared to 2-dimensional cell culture. Lower expression levels of MMP1 and MMP13 indicate healthier tissue than higher expression levels of MMP1 and MMP13. Similarly, scaffold structure may be sufficient to induce the differentiation of exogenous stem cell populations. For example, human mesenchymal stem cells cultured in aligned anisotropic scaffolds exhibited more robust expression of SCX as compared to non-aligned scaffolds. Higher levels of SCX expression suggest differentiation towards mature tenocytes.

Additionally, a scaffold of the invention having a relative density of 0.0156±0.0009, a transverse pore size of 230.4 μm±36.7 μm, a transverse pore aspect ratio of 1.15±0.01, and a longitudinal pore aspect ratio of 1.55±0.25 (“scaffold A”) had different results than a scaffold of the invention having a relative density of 0.0109±0.0003, a transverse pore size of 232.0 μm±14.8 μm, a transverse pore aspect ratio of 1.16±0.06, and a longitudinal pore aspect ratio of 1.72±0.14 (“scaffold B”). For example, scaffold A had higher expression of SCX, MMP3 by equine tenocytes than scaffold B and lower expression of MMP1 and MMP13 by equine tenocytes than scaffold B.

Together, these results suggest that scaffold relative density not only had significant importance in regulating traditional measures of tenocyte bioactivity (attachment, proliferation, metabolic activity, collagen synthesis), but that the degree of anisotropy within a 3D biomaterial microenvironment plays a significant role in regulating the differentiation of human mesenchymal stem cells towards mature tenocytes as well as the transcriptomic stability of mature tenocytes. Scaffold anisotropy can play a significant role in a variety of other tissue engineering applications where the native tissue exhibits a significant degree of microstructural alignment; additionally, the orientation dependent microstructural and mechanical cues available to individual cells within an anisotropic scaffold structure can also have significant importance in regulating stem cell differentiation processes for those same tissues.

### Membrane Shell and Core Composites

A membrane shell and scaffold core composite can have a diameter or thickness of about 1 mm to about 20 mm or any range or value between about 1 mm and about 20 mm. A membrane shell and scaffold core composite can have a length of about 1 mm to about 100 mm or any range or value between about 1 mm and about 100 mm.

The duration of a shell and core composite of the invention is the length of time required for the composite to remain in a relatively solid-like form to, for example, give the composite time function to, for example regenerate tendon at a wound or defect site. The duration of a composite can be about 7 days, 10 days, 2 weeks, 3 weeks, 4 weeks, 5 weeks, 6 weeks, 7 weeks, 8 weeks, 3 months, 4 months, 5 months, six months, 1 years, 2 years or more (or any range or value between about 7 days to about 2 years, for example about 4 weeks to about 6 months).

In relation to the membrane shell, core and composite parameters discussed above, the term “about” means that the stated parameter value can vary by 5% or less.

### Methods of Making Composites

A membrane shell can be fabricated from a CG suspension via an evaporative process. Degassed CG suspension is pipetted onto a solid, flat surface and allowed to dry at room temperature. Alternatively, the suspension can be pipetted onto a solid surface containing surface topology or raised features of sufficient depth to alter the local thickness of the membrane or create perforations in the completed membrane. These features may be of the scale of about 10 μm to about 5 mm (or any range or value between about 10 μm to about 5 mm, for example from about 100 μm to about 750 μm, or about 500 μm. Optionally, the membrane shell can be only partially dried. A CG membrane shell is cut to size, rolled, and placed within a mold, for example, a cylindrical PTFE copper mold. The CG membrane is placed in the mold so that it contacts the longitudinal surfaces of the mold leaving the center or core open for the addition of the scaffold core portion of the composite. There may be perforations in the membrane shell such that certain parts of the CG membrane do not contact or cover every surface of the mold. A CG scaffold suspension is then pipetted into the rolled CG membrane shell, which is within the mold. The CG scaffold suspension can be allowed to hydrate the CG membrane shell for about 5, 10, 15, 20, 45, 60, 90, 120 minutes or more. The hydration can help improve attachment of the membrane shell to the scaffold core. The mold is then placed into a freeze dryer at a pre-cooled temperature (e.g., about −10° C. to about −60° C.) to promote directional solidification of the scaffold core. After freezing, ice crystals can be sublimated under vacuum to produce a scaffold core with aligned pores surrounded by a membrane shell. The membrane-shell scaffold core composite is removed from the mold to form a non-cross-linked composition. The composition can optionally be sterilized and then cross-linked, e.g., by dehydrothermal cross-linking and/or carbodiimide chemistry using any suitable method, e.g., 1-ethyl-3-[3-dimethylaminopropyl]carbodiimide hydrochloride (EDAC) and N-hydroxysulfosuccinimide cross-linking or gluteraldehyde cross-linking, to form a core-shell composite. The composites can be stored hydrated (i.e., PBS, distilled water) or dried prior to use.

### Methods of Use of Composites

Defects in any tissue having an aligned morphology, e.g., bone, tendon, cartilage, ligament, muscle, cardiac tissue, connective tissue, nerve tissue (peripheral and central). Composites of the invention can be used in the treatment of or prevention of, e.g., a bone fracture or bony defects or injuries, cartilage defects or injuries, tendon defects or injuries, ligament defects or injuries, muscle defects or injuries, cardiac tissue defects and injuries, and nerve (peripheral or central) defects or injuries, or any other tissue exhibiting an aligned morphology. Tendon defects and injuries include tendinopathies or tendon injuries due to overuse, tendon rupture, paratenonitis, tendinosis, paratenonitis with tendinosis, tendinitis. Ligament defects and injuries include ligament degradation due to inflammation, damage or degradation due to rheumatoid arthritis, mixed connective tissue disease, polycondritis, systemic lupus erythematosus and scleroderma; infection; overstretched or torn ligaments; ligament avulsion.

“Treatment” or “treating” refers to administration or application of a composite of the invention to a subject or performance of a procedure on a subject using a composite of the invention for the purpose of obtaining a therapeutic benefit of a disease or health-related condition.

The term “therapeutic benefit” or “therapeutically effective” is the promotion or enhancement of the well-being of the subject with respect to the medical treatment of a condition. This includes, but is not limited to, a reduction in the frequency or severity of the signs or symptoms of a disease or injury.

“Prevention” and “preventing” refers to administration or application of composite of the invention to a subject or performance of a procedure using a composite of the invention on a subject to block or slow the onset of a disease or health-related condition. For example, a composite of the invention can be used to prevent connective tissue or bone disease in a subject. The composites of the invention can, in certain embodiments, be utilized as an implant for a therapeutic benefit. In particular embodiments, the composites can be used for connective tissue or bone augmentation. In certain embodiments, composites of the invention are shaped to duplicate connective tissue or bone lost by a subject. Composites shaped in this matter can, for example, be implanted in the subject such that the body may regenerate bone or connective tissue to replace the lost matter.

In one embodiment of the invention one or more therapeutic agents can be integrated into the scaffold core or membrane shell or both the scaffold core and membrane shell. Therapeutic agents can be, e.g., one or more biomolecules such as enzymes, receptors, neurotransmitters, hormones, cytokines, cell response modifiers such as growth factors and chemotactic factors, antibodies, vaccines, haptens, toxins, interferons, anti-sense agents, plasmids, DNA, RNA, anti-cancer substances, antibiotics, anti-inflammatory agents, immunosuppressants, anti-viral agents, enzyme inhibitors, neurotoxins, opioids, hypnotics, antihistamines, lubricants, tranquilizers, anti-convulsants, muscle relaxants, antispasmodics, antifungal agents, cell growth inhibitors, anti-adhesion molecules, vasodilating agents, inhibitors of DNA, RNA, or protein synthesis, antihypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, prostaglandins, chemotactic factors, proteins, cells, peptides, glycoprotein, lipoprotein, steroidal compound, vitamin, carbohydrate, lipid, extracellular matrix component, chemotherapeutic agent, anti-rejection agent, viral vector, protein synthesis co-factor, endocrine tissue, collagen lattice, cytoskeletal agent, fibronectin, growth hormone cellular attachment agent, surface active agent, hydroxyapatite, penetration enhancer, laminin, fibrinogen, vitronectin, trombospondin, proteoglycans, decorin, proteoglycans, beta-glycan, biglycan, aggrecan, veriscan, tanascin, chemokines, interleukines, tissue or tissue fragments, endocrine tissue, collagenase, peptidases, oxidases; bioadhesives; bone morphogenic proteins (BMPs), transforming growth factors (TGF-β), insulin-like growth factor, platelet derived growth factor (PDGF), fibroblast growth factors (FGF), vascular endothelial growth factors (VEGF), epidermal growth factor (EGF), and growth factor binding proteins, e.g., insulin-like growth factors.

In one embodiment of the invention a therapeutic agent is platelet-derived growth factor BB (PDGF-BB), insulin-like growth factor 1 (IGF-1), basic fibroblast growth factor (bFGF), stomal cell-derived factor 1α (SDF-1α), growth/differentiation factor 5 (GDF-5), growth/differentiation factor 7 (GDF-7), or combinations thereof. A therapeutic agent can be added as a soluble agent to the composites of the invention or immobilized (permanently or temporarily) to the composites of the invention (to the membrane shell, core or both the membrane shell and core). In one embodiment of the invention, PDGF-BB is added at about 1 to about 500 ng/ml (or any range or value between about 1 to about 500 ng/ml) of media when delivered as a soluble agent; IGF-1 and SDF-1α are added at about 1 to about 1,000 ng/ml (or any range or value between about 1 to about 1,000 ng/ml) of media when delivered as a soluble agent; bFGF is added at about 0.01 to about 100 ng/ml (or any range or value between about 0.01 to about 100 ng/ml) of media when delivered as a soluble agent; GDF-5 is added about 1 to about 2,000 ng/ml (or any range between about 1 and about 2,000 ng/ml) of media when delivered a soluble agent.

When immobilized to any portion of a composite PDGF-BB, IGF-1, bFGF, SDF-1α, and/or GDF-5 can be present at about 0.0001 μg/mm³ to about 10 μg/mm³ of composite (or any range or value between about 0.0001 μg/mm³ to about 10 μg/mm³) of composite.

The addition of PDGF-BB, IGF-1, bFGF, SDF-1α, and/or GDF-5 to a composite can increase cell, e.g., tenocyte, migration to a composite, motility, increase cell number in the composite, viability, and/or increase metabolic activity in the composite in a dose-dependent manner. While any combinations of therapeutic agents can be added to the composite as a soluble factor or immobilized to the composite, in one particular embodiment both IGF-1 and GDF-5 are added in combination to a composite.

One or more therapeutic agents may be coated or immobilized (permanently or temporarily) onto the membrane shell, and/or scaffold core, incorporated into the membrane shell and/or scaffold core, incorporated into microspheres that are distributed in the membrane shell and/or scaffold core, or the membrane shell scaffold core composite can be immersed in a composition comprising one or more therapeutic agents prior to use in vitro implantation into a subject.

In one embodiment of the invention one or more therapeutic agents, e.g. biomolecules, can be immobilized to a membrane shell and/or scaffold core by a photolithography-based sequestration of the agents. [40]. Briefly, benzophenone is added to the collagen-glycosaminoglycan core or scaffold or core shell composite in the dark. The one or more therapeutic agents are added to one or more areas of the collagen-glycosaminoglycan core or scaffold or core shell composite in the dark; and the core or scaffold or core shell composite is exposed to light at a wavelength of about 350 to about 365 nm. One or more portions of the scaffold, core, or core shell composite can be exposed to the light while one or more other portions remain in the dark. The one or more types of biomolecules can be immobilized onto the core, scaffold, or core shell at two or more different depths.

The invention provides methods for repairing injured, diseased, ruptured or damaged tissue with aligned structure, such as bone, cardiac tissue, muscle tissue, nerve tissue (peripheral or central), or connective tissue, such as a ligament, meniscus, rotator cuff, nerve, skin, cartilage, or tendon. The method comprises positioning a first end of a composite of the invention adjacent a first end of a defective tissue; positioning a second end of the composite adjacent a second end of the defective tissue; wherein the composite provides a scaffold for cell growth and tissue repair. One or more bioactive or therapeutic agents that can stimulate cell growth and tissue repair can be administered to the area.

The positioning the first end and the second end of the composite can further comprise anchoring the first end of the composite to the first end of the defective tissue, and anchoring the second end of the composite to the second end of the defective tissue.

Composites of the invention can be used in a variety of surgical and non-surgical applications. For surgical applications, compositions can be sutured or otherwise fastened to tissue without tearing. Suitable mechanical fasteners include, for example, sutures, staples, tissue tacks, suture anchors, darts, screws, pins and arrows. A composite can also be affixed to a subject by a chemical fastening technique. Chemical fasteners include, for example, glues or adhesives such as fibrin glue, fibrin clot, and other known biologically compatible adhesives. A combination of one or more chemical fasteners and/or mechanical fasteners can be used. Alternatively, chemical or mechanical fasteners are not used. Instead, placement of the composite can be accomplished fitting of the composite into an appropriate site in the tissue to be treated.

Tissue can be grow on the surface of the composite, or alternatively, tissue can be grown into and on the surface of the composite, such that the tissue becomes embedded in and integrated with the composite.

A composite of the invention can be used for repair and to augment tissue loss during connective tissue, bone or other tissue repair surgery or it can be used as a stand-alone device. In the case of repair, tissue ends are approximated through appropriate surgical techniques and the composite is used around the joined end to give more mechanical support and to enhance the healing response. During healing, the tissue grows within the composite, eventually maturing into a tissue with similar mechanical properties to that of native tissue. The composite provides mechanical support necessary to ensure proper healing, and also serves as a guide for tissue regeneration. For stand-alone use, the defective tissue is removed, and the composite, optionally seeded with appropriate cells serves to replace the defective tissue. The defective tissue can be used as the cell source used for seeding the composite prior to implantation.

Composites of the invention can be used for tissue augmentation in ligament or tendon tissue repair procedures. Composites can be used in conjunction with any of a variety of standard, established ligament repair techniques. For example, during ACL repair, an autograft consisting of ligament tissue, bone-patellar tendons, tendon-bone tendons, hamstring tendons, or iliotibial band can be used to repair tissue. Composites of the invention can be placed around the autograft, can be surrounded by the autograft, or placed alongside the autograft to augment the repair. Alternatively, a defective ligament or tendon can be removed and completely replaced by a composite. In this case, the composite can be affixed to bone or muscle at each end of the implant. In the case of ACL repair, one end of the implant can be stabilized at the original origin site of the femur, while the other end can be placed at the original insertion site on the tibia.

The composite can be used to repair tendons, for example, rotator cuff. A composite can be used to assist in the reapproximation of the torn rotator cuff to a bony trough through the cortical surface of the greater tubercle. Rotator cuff tissue can be thin and degenerate and/or the quality of the humerus can be osteoporotic. In these cases the strength of the attachment to the bony trough can be increased by placing the composite on top of the tendon, such that the sutures pass through both the scaffold and tendon, or alternatively, the composite can be used on top of the bone bridge to prevent the sutures from pulling out of the bone. In either case, the composite provides suture retention strength. Where the rotator cuff cannot be reapproximated to the humerus, a composite can serve as a bridge, where one end of the composite can be joined to the remaining tendon while the other end can be attached to the bone.

### Kits

The invention provides kits that include one or more a membrane shell core composites. The membrane shell core composites can be sterilely packaged. In the kit, the membrane shell core composites can be in an appropriate medium such as PBS. Optionally, the core-shell composite can be dried or partially dried and present in a storage container suitable for preserving the core-shell composite until use. The kits can further include one or more therapeutic agents that can be administered concurrently or consecutively with implantation of the composite. The kits can include hardware for placement of the composite in the subject, or a device for further shaping the composite into a desired configuration.

All patents, patent applications, and other scientific or technical writings referred to anywhere herein are incorporated by reference herein in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### CG Membrane Fabrication

CG suspensions were prepared from type I microfibrillar collagen (0.5% w/v) isolated from bovine dermis (Devro Inc., Columbia, S.C.) and chondroitin sulfate (0.05% w/v) derived from shark cartilage (SigmaAldrich, St. Louis, Mo.) in 0.05 M acetic acid [19]. The suspension was homogenized at 4° C. to prevent collagen gelatinization during mixing and was subsequently degassed before use.

CG membranes were fabricated from the CG suspension via a modified evaporative process [26]. Briefly, the degassed CG suspension was pipetted into a Petri dish and allowed to air dry in a chemical fume hood at room temperature for 2-3 days. In order to create a series of CG membranes of variable thickness, a series of membranes were fabricated via the identical method but using CG suspension of different volumes (25-50 mL) and/or densities (0.5% w/v, 1% w/v). The primary membrane variants tested were 0.5% w/v 25 mL, 0.5% w/v 50 mL, 1% w/v 25 mL, and 1% w/v 50 mL. Another membrane was fabricated by sequential addition of 1% w/v CG suspension to the same Petri dish (150 mL total volume).

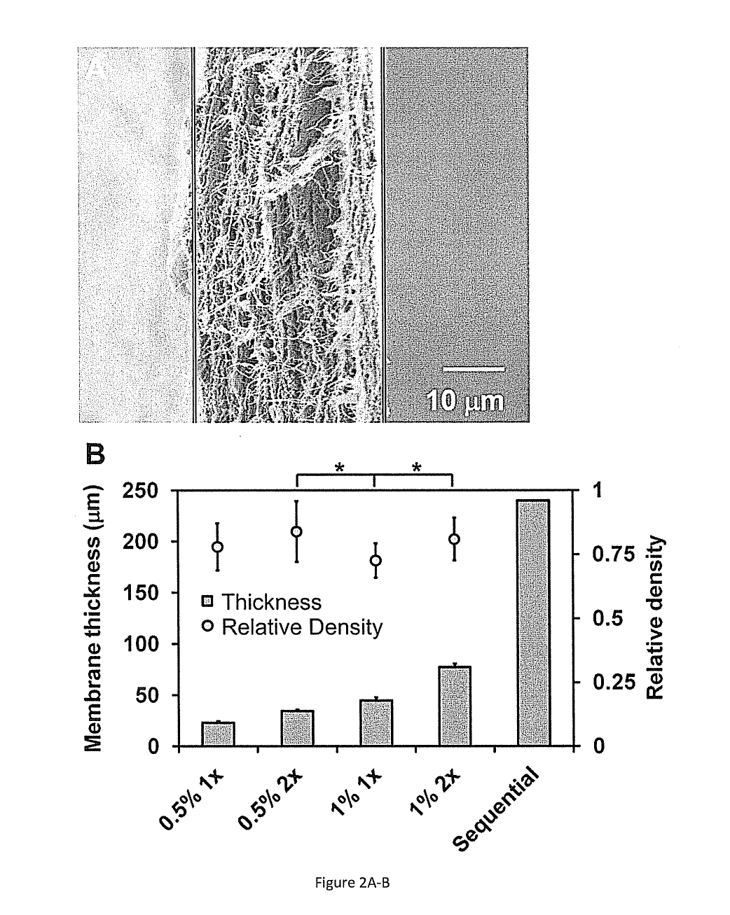

CG membranes consistently displayed a dense network of fibrillar collagen content (FIG. 2(A)). The thickness of the final membrane was observed to increase with either the collagen-GAG (glycosaminoglycan) wt % in the CG suspension or with the volume of suspension used (FIG. 2(B)). The experimental groups were created from either 0.5% w/v or 1% w/v CG suspensions with either 1× volume (25 mL) or 2× volume (50 mL) of suspension added to the Petri dish prior to drying in order to create four membrane variants: 23±1, 35±1 μm (0.5% w/v suspension; 1×, 2× volume); 45±3 μm, 78±3 μm (1% w/v suspension; 1×, 2× volume). Additionally, sequential (n=6, 1% w/v suspension) addition of CG suspension to the same Petri dish during the process of evaporative drying was used to create membranes as thick as 240 μm.

All CG membranes were found to possess consistent relative densities between 0.75 and 0.80 (20-25% porous) (FIG. 2(B)). While statistically significant differences in membrane relative density were observed between some groups (1% w/v 1× vs. 0.5% w/v 2×, p=0.003; 1% w/v 1× vs. 1% w/v 2×, p=0.009), these differences do not suggest any trend. Swelling assays revealed that all four membrane variants tested (0.5% w/v 1×, 0.5% w/v 2×, 1% w/v 1×, and 1% w/v 2×) showed consistent hydration curves and were at least 90% hydrated after 30 min in PBS (data not shown).

### Aligned CG Scaffold and Composite Fabrication

Aligned CG scaffolds (ρ*/ρₛ=0.006) were fabricated [23]. Briefly, the CG suspension was added to wells of a multicomponent polytetrafluoroethylene (PTFE)-copper mold, and placed on a freeze-dryer shelf (VirTis Genesis, Gardiner, N.Y.) at a pre-cooled temperature (−10° C. or -60° C.) in order to promote directional solidification. After freezing, ice crystals were sublimated under vacuum (200 mTorr) at 0° C. to produce CG scaffolds (6 or 8 mm diameter, 15 or 30 mm length) displaying aligned pores (−10° C.: 243±29 mm; −60° C.: 55±18 mm) [23]. Mechanical tests were performed on 6 mm diameter by 30 mm length scaffolds to facilitate placement of the constructs within the mechanical tester grips. Scaffold-membrane constructs were fabricated by first cutting CG membrane pieces to size and placing circumferentially within the PTFE-copper mold. The CG suspension was then pipetted inside the rolled membrane and allowed to hydrate the membrane for ˜15 min at 4° C. before the mold was placed into the freeze-dryer held at a final freezing temperature of −10° C.; freezing and sublimation steps for these scaffold-membrane constructs were performed exactly as with the anisotropic scaffolds alone. Membrane hydration and subsequent freeze-drying was hypothesized to promote the integration of the scaffold structure with the membrane [24].

We describe an evaporative process to fabricate CG membranes with tailorable thicknesses over an order of magnitude (23-240 μm), but consistent relative densities of ˜0.75-0.80 that are significantly higher than those of the CG scaffold (0.006) (FIG. 2(B)). CG membranes were mechanically isotropic in-plane, and as with CG scaffolds [12,19] increasing the degree of physical (DHT) or chemical (carbodiimide) cross-linking significantly increased membrane tensile moduli (FIG. 2(A-B)). The EDAC 5:2:1 groups displayed ˜7-10 fold increases in modulus over noncross-linked controls, comparable in magnitude shift to previous work with CG scaffolds [12]. Additionally, CG membranes were observed to swell with similar kinetics as observed for CG scaffolds [20] and to reach an asymptote, suggesting a stable membrane structure.

### Cross-Linking

Scaffolds, membranes, and scaffold-membrane composites were sterilized and dehydrothermally (DHT) cross-linked at 105° C. for 24 h under vacuum (<25 torr) in a vacuum oven (Welch Vacuum Technology, Niles, Ill.) prior to use [12,19]. Scaffolds and composites were then immersed in 100% ethanol overnight, washed with phosphate-buffered saline (PBS) several times over 24 h, and then cross-linked using carbodiimide chemistry [12,40] for 1 h in a solution of 1-ethyl-3-[3-dimethylaminopropyl]carbodiimide hydrochloride (EDAC) and N-hydroxysulfosuccinimide (NHS) at a molar ratio of 5:2:1 EDAC:NHS:COOH. To test the effect of cross-linking density on membrane mechanics, some membranes were hydrated directly in PBS without further cross-linking (Non-cross-linked, NC) or were cross-linked using EDAC chemistry at a molar ratio of either 1:1:5 or 5:2:1 EDAC:NHS:COOH. Scaffolds, membranes, and composites were subsequently stored in PBS until use.

### Determination of Membrane Microstructural Properties

Qualitative analysis of scaffold, membrane, and scaffold-membrane composite microstructure was performed using scanning electron microscopy (SEM). SEM analysis was performed with a JEOL JSM-6060LV Low Vacuum Scanning Electron Microscope (JEOL USA, Peabody, Mass.) using both a standard secondary electron (SE) detector and a backscatter electron (BSE) detector under low vacuum mode, bypassing the need for any sample sputter coating steps [24].

Membrane thickness was determined from cross-sectional SEM images. Six 500× magnification images were taken for each membrane type, and the thickness of the membrane was measured using a multipoint measuring tool within the SEM software. The relative density (ρ*/ρₛ) of each CG membrane type was determined from the calculated density of the membrane (ρ*) relative to the known density of solid collagen (ρₛ, 1.3 g cm⁻³) [12,28,29]. Swelling kinetics and final swelling ratio of each CG membrane variant was determined by monitoring the weight of 2 cm by 2 cm membranes samples (n=6) hydrated in PBS for 5, 10, 15, 30 and increasing 30 min intervals up to 240 min. A normalized swelling curve was calculated for each membrane variant and the time required for complete hydration was defined as the point where the curve reached a plateau [30].

### Mechanical Characterization

Tensile tests were performed on CG membranes (12 mm width, 45 mm length), aligned scaffolds (6 mm diameter, 30 mm length), and core-shell scaffold membrane composites (6 mm diameter, 30 mm length). Tensile tests were performed in a manner consistent with previous mechanical analysis of CG scaffolds [12,31]. Specimens were hydrated in PBS for 24 h prior to testing and were then pulled to failure at a rate of 1 mm/min using an MTS Instron 2 (Eden Prairie, Minn.) with rubberized grips to prevent slip. Tensile modulus was calculated from the slope of the stress-strain curve over a strain range of 5-10% in the case of scaffolds and composites [9] and over the initial linear region for membranes [31]. For comparison to the anisotropic scaffolds, previously reported mechanical data for an isotropic control CG scaffold with a consistent relative density was used [32]. All samples tested were hydrated in PBS unless otherwise specified. Layered composites theory was applied to predict the tensile modulus (E*_{composite}) of the final composites from the relative size and modulus of scaffold (composite radius, r; (E*_{scaffold}) and membrane (membrane thickness, t; (E*_{composite}) components and their separate moduli [51]:

$${E}_{composite}^{*} = {E}_{scaffold}^{*} ( \frac{{( r - t )}^{2}}{{r}^{2}} ) + {E}_{membrane}^{*} ( 1 - \frac{{( r - t )}^{2}}{{r}^{2}} ) \qquad ( Equation 1 )$$

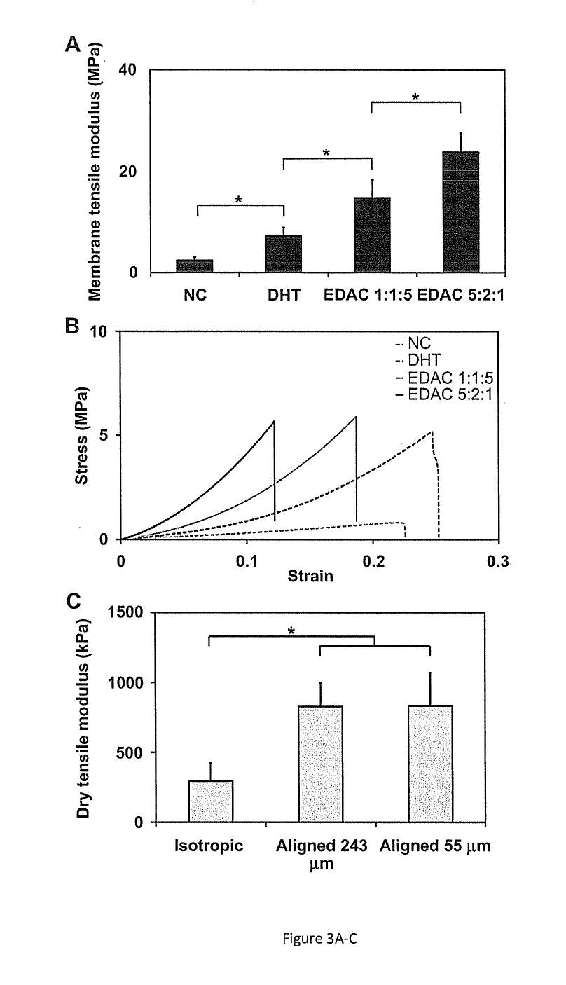

As expected due to random evaporative processes, the CG membranes were found to be isotropic in-plane. Dry specimens from 1% w/v 1× volume membranes were cut into samples from orthogonal directions (‘parallel’ vs. ‘perpendicular’ samples) and then pulled to failure. The tensile modulus of the dry membranes in the perpendicular orientation (636±47 MPa) was not significantly different from the parallel orientation (693±20 MPa) (p=0.06). Like with CG scaffolds, the tensile modulus of the CG membranes was found to increase significantly with cross-linking treatment and intensity [12]. The tensile moduli of hydrated CG membrane specimens (0.5% w/v 1×, 1% w/v 1×) was determined after no cross-linking (NC), dehydrothermal cross-linking (DHT), DHT plus carbodiimide cross-linking at a 1:1:5 EDAC:NHS:COOH molar ratio (EDAC 1:1:5), and DHT plus carbodiimide cross-linking at a 5:2:1 M ratio (EDAC 5:2:1). For the 0.5% w/v membranes, no significant difference was observed between the NC and DHT groups (p=0.86), but significant differences were observed between all other groups (p≦0.01, all groups). The tensile modulus of the hydrated 1% w/v 1× membranes was found to significantly increase with each increasing cross-linking step (p≦0.02, all groups), resulting in hydrated CG membranes showing tensile moduli approaching 30 MPa, multiple orders of magnitude stiffer than CG scaffolds (FIG. 3(A-B)). While there is a notable decrease in membrane modulus after hydration, this is consistent with previous results for CG scaffolds [12].

All scaffolds displayed a consistent relative density (0.6%) [23]. The aligned CG scaffold variants (Pore sizes: 55±18 μm, 243±29 μm) displayed significantly higher dry tensile modulus (833±236, 829±165 kPa) compared to an isotropic CG control [32] (p≦0.03) (FIG. 3(C)). Cellular solids theory and previous experimental results predict that mechanical properties of a series of scaffolds with constant relative density and mean pore shape (i.e. degree of anisotropy) will be independent of pore size [9,12]. While slight differences in the aspect ratio (A.R., a measure of the degree of pore anisotropy) for the two aligned scaffolds have been noted (55 μm, 1.41±0.16; 243 μm, 1.57±0.23) [23], no difference in scaffold tensile modulus was observed between the anisotropic scaffolds (p=0.96).

TC attachment, proliferation, metabolic activity, and degree of TC populational alignment are critically affected by both scaffold pore size and degree of anisotropy [23]. The effect of scaffold anisotropy on its tensile properties and the capacity of the core-shell paradigm to significantly improve construct properties were investigated. Aligned tissue engineering scaffolds have consistently demonstrated superior mechanical properties along the axis of alignment compared to isotropic controls [5,36], though these results have mainly been shown using 2D electrospun materials or single unit cell thick honeycomb-like structures [37]. We showed that two aligned CG scaffold variants with significantly different pore sizes (55, 243 μm) had tensile moduli nearly three times greater than that of an isotropic CG scaffold control fabricated at the same relative density (FIG. 3(C)). For a series of scaffolds with constant ρ*/ρₛ such as the three scaffolds tested here, two predictions regarding scaffold mechanical properties were made. First, that scaffold modulus is a function of relative density but not pore diameter. This was confirmed by showing that aligned scaffolds with identical relative densities but different pore sizes had nearly identical tensile moduli (FIG. 3(C)). Second, that scaffold modulus should increase with pore anisotropy (when scaffolds are tested in the direction of anisotropy); this was confirmed by showing the significant increase in anisotropic scaffold modulus relative to the isotropic control (FIG. 3(C)). We then applied cellular solids theory to predict changes in the elastic moduli of anisotropic vs. isotropic open cell foams. Here, the predicted modulus of the anisotropic scaffolds (E*ₐ) can be described with the isotropic scaffold modulus (E*ᵢ) and the an isotropic:isotropic scaffold pore aspect ratios (R):

$${E}_{a}^{*} = {E}_{i}^{*} ( \frac{2 {R}^{2}}{1 + {( 1 / R )}^{3}} ) \qquad ( 2 )$$

Based on the previously reported aspect ratios of the 55 μm and 243 μm aligned scaffolds neglecting the end sections held in the clamps during tensile testing [23] the predicted moduli would be 880 kPa and 913 kPa for the 55 μm and 243 μm scaffolds, comparing favorably to the experimentally achieved values of 833±236 kPa and 829±165 kPa.

Structural Features and Mechanical Properties of Aligned Scaffold-Membrane Core-Shell Composites

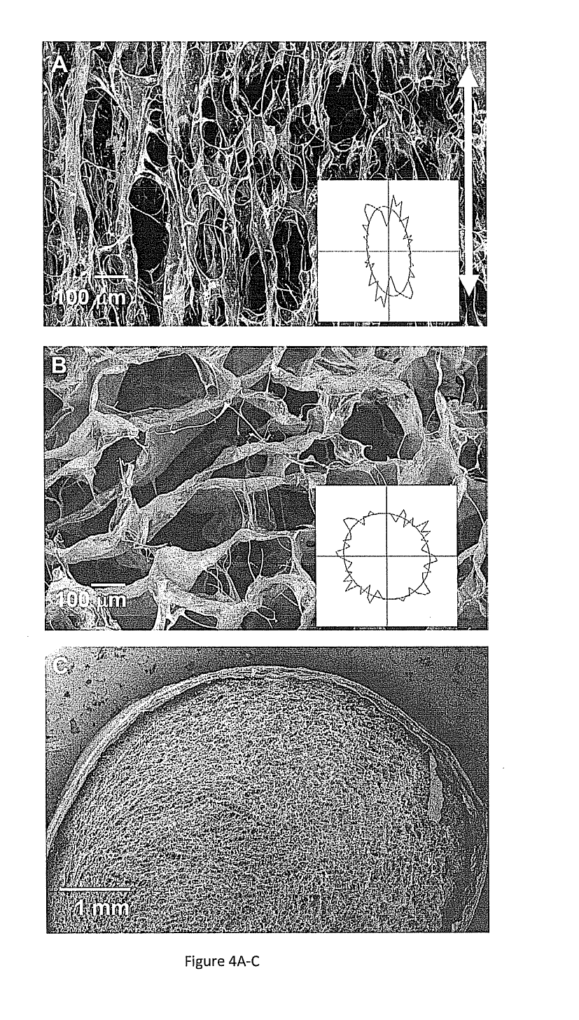

The CG scaffold core of the core-shell composites showed aligned, elongated pores in the longitudinal plane (FIG. 4(A)) and circular, more isotropic pores in the transverse plane (FIG. 4(B)) as a result of unidirectional heat transfer applied during freeze-drying. The CG membrane showed stable integration with the CG scaffold core (FIG. 4(0)), with limited delamination observed during freeze-drying, hydration, cross-linking, or mechanical testing processes.

CG scaffold-membrane core-shell composites were fabricated via liquid-solid phase co-synthesis [24] in a manner aimed at achieving functional integration between CG scaffold and membrane components by allowing a hydration time-step for the CG suspension to hydrate the membrane prior to freezing. SEM confirmed the creation of an aligned microstructure in the longitudinal plane (FIG. 4(A)) while the transverse plane showed a more isotropic structure (FIG. 4(B)), consistent with results reported for the aligned CG scaffold alone [23]. These results indicate that addition of the CG membrane did not adversely influence the directional solidification process required to create the aligned CG scaffold microstructure, and was expected because the CG membrane should not alter the degree of thermal conductivity mismatch (k_{Cu}/k_{PTFE}˜1600) in the composite mold [23]. Importantly, it was demonstrated that the CG membrane could be integrated into the CG scaffold structure to form a continuous composite material (FIG. 4(C)) that exhibited limited delamination during fabrication, structural and mechanical analysis, and in vitro cell culture components of this study. Comparatively, wrapping the membrane around a complete scaffold required gluing or sutures to prevent delamination. The degree of membrane incorporation theoretically can be tuned by adjusting the hydration time of the membrane in the scaffold suspension prior to freeze-drying, presenting a future avenue for testing and development, particularly in the light of the mechanical results discussed below.

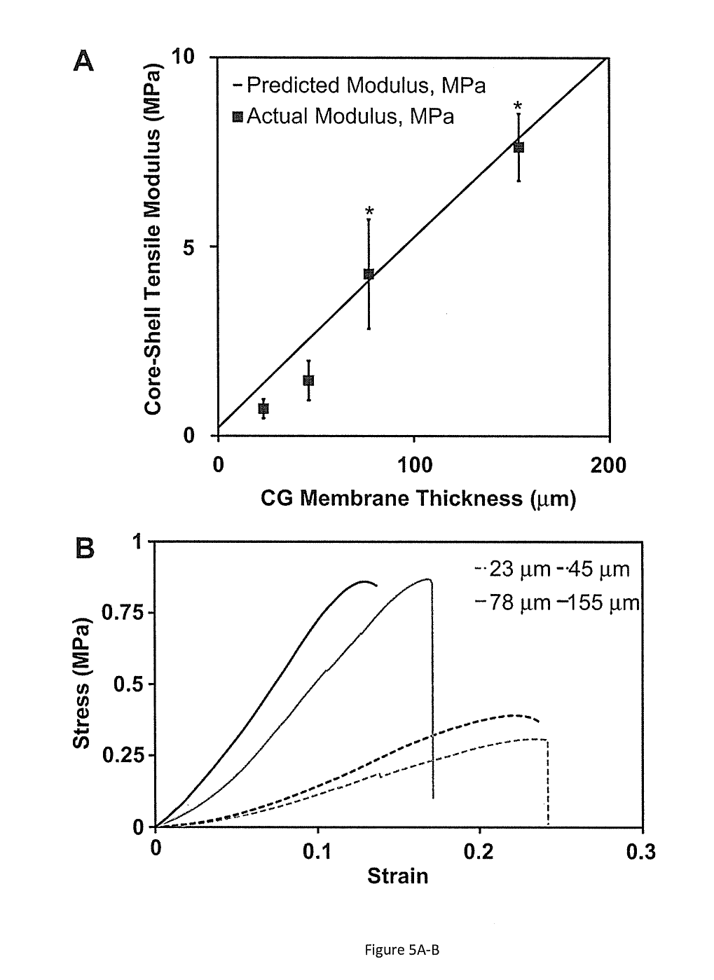

Core-shell composites were created from a consistent aligned CG scaffold (pore size: 243±29 μm) core and one of four 5:2:1 EDAC-cross-linked membranes of distinct thicknesses: 23 μm (0.5% w/v 1×), 45 μm (1% w/v 1×), 78 μm (1% w/v 2×), and 155 μm (1% w/v 2× wrapped twice around scaffold). A significant effect of membrane thickness was observed on the tensile modulus of the aligned scaffold-membrane core-shell composites (p<0.0001). While the increase in modulus between the core-shell composites with 23 μm and 45 μm thick membranes was not significant (p=0.14), significant differences were observed between all other groups (p 0.001) (FIG. 5(A-B)). Scaffold-membrane composites also demonstrated dramatically increased tensile moduli over aligned CG scaffolds alone, with a 36-fold increase observed for the 155 μm membrane composite. Experimental results closely mirrored theoretical predictions (solid line, FIG. 5(A)), indicating that the scaffold core and membrane shell were functionally integrated.

After separately characterizing the mechanical properties of the aligned CG scaffolds and CG membranes, CG scaffold-membrane composites were fabricated and characterized using membranes ranging in thicknesses from 23 μm (0.5% w/v 1×) to 155 μm (1% w/v 2× wrapped twice around scaffold). These composites demonstrated dramatically increased tensile moduli over CG scaffold controls (no membrane shell) with a 36-fold increase observed for the 155 μm thick membrane (FIG. 5). The aspect ratios of the scaffold, membrane, and scaffold-membrane samples tested in tension were consistent within groups. However, because the membrane sample aspect ratios were greater than the scaffold and scaffold membrane samples, it is possible that the extension behavior of the membrane vs. scaffolds was different due to differential stress propagation in the specimens. Experimental results for the scaffold-membrane composite were also compared to predictions from layered composites theory, which has previously been used to accurately predict the tensile properties of multicomponent materials based on the relative size of the individual components and their separate moduli [25].

Experimental results correlated well with theoretical predictions, especially for composites with the two thicker membranes (78 μm, 155 μm). However, the experimental values for tensile moduli of the core-shell composites with the two thinnest membranes (23, 45 μm) fell somewhat short of theoretical predictions from layered composite theory (FIG. 5(A)). These results may suggest a degree of incomplete integration between the core and shell components for the thinnest shell composites, with superior, more complete incorporation observed for the thicker membranes with the scaffold core. Overall, the close agreement of the experimental results with the theoretical predictions as well as the low incidence of composite delamination suggests that the core-shell scaffolds behave like layered composites, implying adequate integration of the membrane with the scaffold.

### Tendon Cell Culture and Bioassays

Tendon cells (TCs) were isolated from horses aged 2-3 years euthanized for reasons not related to tendinopathy [33]. TCs were then expanded in standard culture flasks in high glucose Dulbecco's modified Eagle's medium (DMEM, Fisher Scientific, Pittsburgh, Pa.) supplemented with 10% fetal bovine serum (FBS, Invitrogen, Carlsbad, Calif.), 1% L-glutamine (Invitrogen, Carlsbad, Calif.), 1% penicillin/streptomycin (Invitrogen, Carlsbad, Calif.), 1% amphotericin-B (MP Biomedical, Solon, Ohio), and 25 pg/mL ascorbic acid (Wako, Richmond, Va.) [33]. TCs were fed every 3 days and cultured to confluence at 37° C. and 5% CO₂. After expansion TCs were either frozen (50% DMEM, 40% FBS, 10% DMSO in liquid nitrogen) for later experiments or used (passage 3) for scaffold culture.

CG scaffold pieces (8 mm diameter, ˜5 mm thickness, with and without outer membrane) were cut from the middle section of 8 mm diameter by 15 mm length scaffolds and placed in ultra-low attachment 6 well plates (Corning Life Sciences, Lowell, Mass.). Confluent TCs were trypsinized and resuspended at a concentration of 5×10⁵ cells per 20 μL media. Scaffolds were initially seeded with 10 μL of cell suspension, incubated at 37° C. for 15 min, turned over, and seeded with an additional 10 μL of cell suspension for a total of 5×10⁵ cells per scaffold [23]. Scaffolds were incubated at 37° C. and 5% CO₂ for the duration of all experiments and were fed with complete DMEM that was changed every 3 days.

A DNA quantification assay was used to determine the number of cells attached to the scaffold [23]. Briefly, scaffolds were washed in PBS to remove unattached cells, placed in a papain solution to digest the scaffold and lyse the cells in order to expose their DNA, and then incubated with a Hoechst 33258 dye (Invitrogen, Carlsbad, Calif.) to fluorescently label double-stranded DNA [23,34]. Fluorescence intensities (352/461 nm excitation/emission) from each sample were read using a fluorescence spectrophotometer (Varian, Santa Clara, Calif.) and then compared to a standard curve created from known numbers of TCs. Cell numbers are reported as a percentage of the total number of seeded cells; numbers of attached cells at day 1 were considered to be a measure of initial cell attachment efficiency [21], while cell numbers at subsequent time points were considered a measure of cell proliferation. Cell metabolic activity was determined using a nondestructive alamarBlue assay [23,35]. Cell-seeded scaffolds were incubated at 37° C. in 1× alamarBlue (Invitrogen, Carlsbad, Calif.) solution with gentle shaking for 3 h. Resorufin fluorescence (570/585 nm excitation/emission) was read at using a fluorescence spectrophotometer (Varian, Santa Clara, Calif.) and compared to a standard curve created from known TCs of the same passage as those used in the experiment. Results are expressed as the total metabolic activity of the cells inside the scaffold relative to that of the initially seeded cells. Metabolic activity results were used as a proxy for relative cell health when the total number of attached cells was comparable [23].

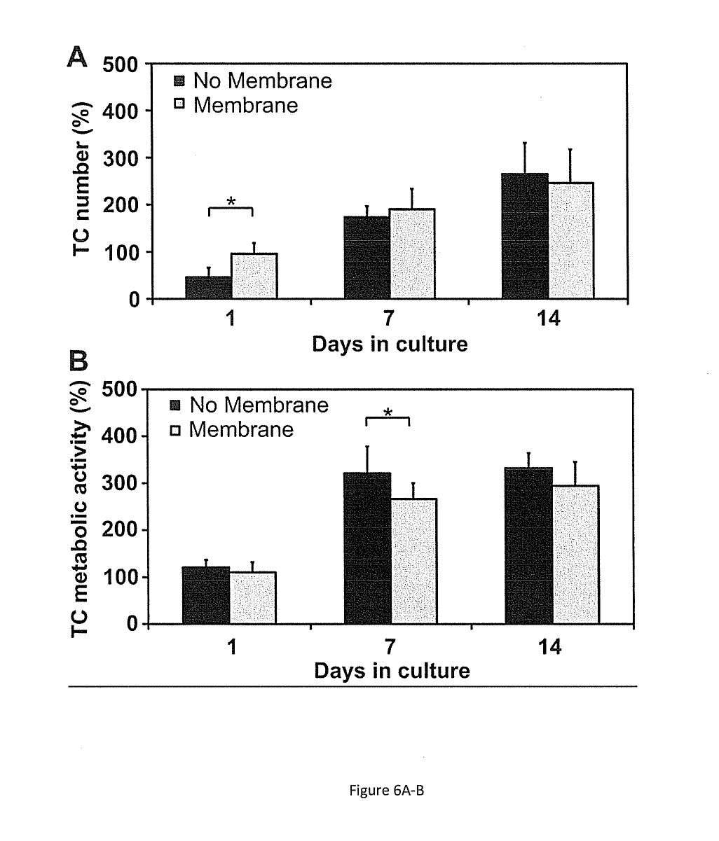

TC number and metabolic activity were assessed over a 14 day in vitro culture period within the aligned CG scaffolds alone (No membrane) or within the core-shell aligned scaffold-membrane composites (Membrane) (FIG. 6); both groups were fabricated with the identical scaffold microstructure (pore size: 243 μm). Early (1 day) results demonstrated that TC number was significantly increased in the core-shell composites (p=0.007) (FIG. 6(A)). While both groups showed increases in TC number over time, no significant differences were observed between the groups at either day 7 (p=0.22) or day 14 (p=0.33). No significant difference was observed in TC metabolic activity at day 1 between the Membrane and No membrane groups (p>0.05) (FIG. 6(B)). While TC metabolic activity in the scaffold alone was significantly higher than that in the core-shell composite at day 7 (p=0.01), TC metabolic activity in the core-shell composites was elevated compared to day 1 and there were no significant differences in metabolic activity between groups at day 14 (p>0.05).

While the scaffold core maintains an open-pore structure conducive for cell penetration and efficient metabolite transport, addition of the CG membrane shell covering ˜75% of the scaffold surface requires assessing cell proliferation and metabolic activity within the composite structure in order to determine its effect on nutrient and oxygen transport into the construct. Typical diffusion distances in CG scaffolds are on the order of 1-2 mm [38], implying the scaffold geometry used here will at minimum provide an environment at its core with reduced metabolite transport. Collagen membranes are typically cell-impermeable but that depending on membrane density can be metabolite and small biomolecule permeable [39]. Therefore, the addition of a 20-25% porous CG membrane shell was not expected to significantly reduce the bioactivity of TCs seeded within the scaffold due to adequate maintenance of metabolite transport. TC number and metabolic activity were measured over a 14 day in vitro culture period in aligned CG scaffold cores (pore size: 243±29 μm) with (Membrane) and without (No membrane) CG membrane shells. After 1 day in culture, the total number of attached TCs was observed to significantly (p=0.007) increase in CG membrane scaffolds relative to the scaffold alone (FIG. 6(A)), with no significant difference (p=0.22) in the metabolic activity (FIG. 6(B)). This result is likely a consequence of the cell-impermeable membrane impacting cell-loss during the seeding step; it is likely that the membrane prevented the cell suspension seeded onto the scaffold from leaking out of the sides, thereby improving cell attachment relative to the scaffold alone where additional cells might be lost. Later time points, a measure of TC proliferation, showed dramatic increases in cell number and metabolic activity compared to day 1 for both groups (FIG. 6(A)). TC number and metabolic activity increased for both groups from day 1 to day 7 and showed further increases at day 14, with no significant differences observed between the groups at this final time point (FIG. 6(A-B)). These results indicate that the core-shell composites have adequate permeability to support the nutrient and metabolite transport necessary for sustained TC viability and proliferation.

The membrane design presented here has adequate permeability to maintain long-term cell viability in vitro, but it is cell impermeable. While adequate TC proliferation and metabolic activity was observed, this was for the case where cells were seeded onto either end of the construct for in vitro assays. For acellular in vivo deployment into a tendon defect, cell penetration from all directions can be facilitated by periodically perforating CG membrane with large (250-500 μm) openings to facilitate radial cell penetration.

### Statistical Analysis

One-way analysis of variance (ANOVA) was performed on membrane and mechanical data sets followed by Tukey-HSD post-hoc tests. Paired student t-tests were used to compare the two groups in cell viability experiments. Significance was set at p<0.05. At least n=6 scaffolds or membranes were used for all analyses. Error is reported in figures as standard deviation unless otherwise noted.

### CONCLUSION

This invention provides CG scaffold membrane (core-shell) composites for connective tissue (e.g., tendon tissue), cardiac, or nerve (peripheral, central), or bone engineering with the intent to avoid aspects of the typical tradeoff between mechanical properties (i.e. modulus, failure strength) and bioactivity (permeability and porosity) for porous tissue engineering scaffolds. Cellular solids modeling provides the framework to describe the tradeoff between mechanical properties and bioactivity proxies (specific surface area, permeability, steric hindrance) as a function of scaffold relative density [9,12,13,21,22,23]. Namely, with increasing scaffold ρ*/ρₛ, modulus (E˜(ρ*/ρₛ)²) and specific surface area (SA/V˜(ρ*/ρₛ)^{0.5}) increase, but permeability decreases (k˜(1−ρ*/ρₛ)^{1.5}) and steric hindrance to cell penetration increases. These relations also predict that to increase CG scaffold elastic modulus by the ˜2 orders of magnitude necessary to achieve levels suitable to prevent mechanical failure in the case of in vivo connective tissue applications, the scaffold relative density would have to be increased from 0.006 to 0.05-0.15. An increase of this magnitude would result in sharp declines in both porosity, permeability, and the ability of cells to penetrate into the scaffold microstructure. The resultant decrease in bioactivity would likely make the scaffolds unsuitable for connective tissue engineering, therefore, the instant core-shell composites were developed. Taking inspiration from mechanically efficient core-shell structures in nature, we felt the scaffold-membrane composite paradigm would provide an alternative strategy to overcome these limitations.

The core-shell CG biomaterial composites of the invention successfully integrate a high density outer shell (isotropic CG membrane) with a low density porous core (anisotropic CG scaffold). The membrane thickness can be controlled over a wide range and the composite Young's modulus can be predicted by layered composites theory. The addition of a membrane shell significantly increases the core-shell composite tensile modulus in a manner consistent with layered composite theory. This invention allows the circumvention of a conventional limitation in biomaterial scaffolds design where construct mechanical strength and porosity are inversely related. Further, these composites also demonstrate the capability to support TC attachment, proliferation, and viability out to 14 days at comparable levels to CG scaffolds alone, indicating CG membranes possess adequate permeability to support cell bioactivity within the scaffold structure.

### REFERENCES

[1] James et al., Tendon: biology, biomechanics, J Hand Surg-Am 2008; 33A:102-12.
[2] Liu et al., Trends Biotechnol 2008; 26:201-9.
[3] Butler et al., J Orthop Res 2008; 26:1-9.
[4] Li et al., Nano Lett 2009; 9:2763-8.
[5] Moffat et al., Tissue Eng Part A 2009; 15:115-26.
[6] Pham et al., Tissue Eng 2006; 12:1197-211.
[7] Sahoo et al., Biomaterials 2010; 31:2990-8.
[8] Altman et al., Biomaterials 2002; 23:4131-41.
[9] Gibson et al., Cellular materials in nature and medicine. Cambridge, U.K: Cambridge University Press; 2010.
[10] Gibson, J Biomech 2005; 38-377-99.
[11] Harley & Gibson, Chem Eng J 2008; 137:102-21.
[12] Harley et al., Acta Biomater 2007; 3:463-74.
[13] O'Brien et al., Technol Health Care 2007; 15:3-17.
[14] Yannas et al., Philos T R Soc A 2010; 368:212339.
[15] Juncosa-Melvin et al., Tissue Eng 2006; 12:2291-300.
[16] Saber et al., Tissue Eng Part A 2010; 16:2085-90.
[17] Chokalingam et al., Tissue Eng Part A 2009; 15:2561-70.
[18] Kuo et al., Tissue Eng Part A 2008; 14:1615-27.
[19] Yannas et al., Proc Natl Acad Sci USA 1989; 86:933-7.
[20] Harley et al., Cells Tissues Organs 2004; 176:153-65.
[21] O'Brien et al., Biomaterials 2005; 26:433-41.
[22] Harley et al., Biophys J 2008; 95:4013-24.
[23] Caliari & Harley BAC, Biomaterials 2011; 32:5330-40.
[24] Harley et al., J Biomed Mater Res A 2010; 92:1078-93.
[25] Allen, Analysis and design of structural sandwich panels. 1st ed. New York: Pergamon Press; 1969.
[26] Pins et al., FASEB J 2000; 14:593-602.
[27] Olde Damink et al., Biomaterials 1996; 17:765-73.
[28] Yannas & Tobolsky, Nature 1967; 215:509-10.
[29] Yannas et al., J Biomed Mater Res 1980; 14:107-32.
[30] Vickers et al., Tissue Eng 2006; 12:1345-55.
[31] Freyman et al., Biomaterials 2001; 22:2883-91.
[32] Kanungo & Gibson, Acta Biomater 2010; 6:344e53.
[33] Kapoor et al., Acta Biomater 2010; 6:2580-9.
[34] Kim et al., Anal Biochem 1988; 174:168-76.
[35] Tierney et al., J Biomed Mater Res A 2009; 91:92-101.
[36] Shang et al., Eur Cells Mater 2010; 19:180-92.
[37] Engelmayr et al., Nat Mater 2008; 7:1003-10.
[38] Harley & Yannas IV. In vivo synthesis of tissues and organs. In: Lanza R, Langer R, Vacanti J P, editors. Principles of tissue engineering. 3rd ed. Elsevier/Academic Press; 2007.
[39] Kimura et al., Tissue Eng Part C Methods 2008; 14:47-57.
[40] Martin et al., Biomaterials 2011; 32:3949-57

We claim:

1. A core-shell composite comprising a porous collagen glycosaminoglycan scaffold core and a collagen glycosaminoglycan membrane shell having a higher density than the core, wherein the membrane shell is cross-linked to the core.

2. The core-shell composite of claim 1 wherein the relative density of the collagen glycosaminoglycan scaffold core is about 0.5 to about 0.95.

3. The core-shell composite of claim 1 wherein the relative density of the collagen glycosaminoglycan membrane shell is about 0.001 to about 0.2.

4. The core-shell composite of claim 1, wherein the core-shell composite is tubular and the composite has a diameter of about 1 mm to about 20 mm.

5. The core-shell composite of claim 1, wherein the collagen glycosaminoglycan membrane shell is periodically perforated with about 25 to about 1000 μm openings.

6. The core-shell composite of claim 1, wherein the porous collagen glycosaminoglycan scaffold core is populated with cells.

7. The core-shell composite of claim 1, wherein the scaffold core is anisotropic or isotropic.

8. The core-shell composite of claim 1, wherein the membrane shell is isotropic or anisotropic.

9. The core-shell composite of claim 6, wherein the cells are adult or embryonic stem and progenitor cells, induced pluripotent cells, tenocytes, osteoblasts, nerve cells, cardiac cells, myocytes, fibroblasts or combinations thereof.

10. A method of making a core-shell composite comprising:
(a) making a collagen glycosaminoglycan membrane shell by placing a collagen glycosaminoglycan suspension on a solid surface and allowing the suspension to dry or partially dry to form a collagen glycosaminoglycan membrane shell;
(b) placing the collagen glycosaminoglycan membrane shell in a mold so that the longitudinal surfaces of the mold are covered with the membrane shell, leaving a center core portion of the mold open;
(c) placing a collagen glycosaminoglycan suspension in the center core portion of the mold;
(d) placing the mold in a pre-cooled freeze dryer;
(e) sublimating any ice crystals to form an non-cross-linked composition;
(f) removing the non-cross-linked composition from the mold and cross-linking the composition to form a core-shell composite.

11. A method of inducing growth of tissue having an aligned structure comprising contacting the core-shell composite of claim 1 with one or more cell types that are capable of forming tissue having an aligned structure and allowing the cells to grow such that growth of tissue having an aligned structure is induced.

12. The method of claim 11, wherein the tissue having an aligned structure is bone tissue, cardiac tissue, muscle tissue, peripheral nerve tissue, central nerve tissue, connective tissue, ligament tissue, meniscus tissue, rotator cuff tissue, skin tissue, cartilage tissue, or tendon tissue.

13. The method of claim 11, wherein the cells are adult or embryonic stem and progenitor cells, induced pluripotent cells, tenocytes, osteoblasts, nerve cells, cardiac cells, myocytes, fibroblasts or combinations thereof.

14. A method of treating a tissue or defect in a subject in need thereof, comprising administering one or more of the core-shell composites of claim 1 to the subject, thereby treating the tissue defect.

15. The method of claim 14, wherein the tissue defect is a defect of bone tissue, cardiac tissue, muscle tissue, peripheral nerve tissue, central nerve tissue, connective tissue, ligament tissue, meniscus tissue, rotator cuff tissue, skin tissue, cartilage tissue, or tendon tissue.

16. The method of claim 14, wherein the core-shell composite is seeded with one or more types of cells prior to administering the core-shell composite to the subject.

17. A kit comprising the core-shell composite of claim 1, wherein the core-shell composite is immersed in a medium or is dried or partially dried and present in a storage container suitable for preserving the core-shell composite.

18. The kit of claim 17, wherein the core-shell composite is seeded with one or more types of cells.
